(19) United States
(12) Patent Application Publication
(10) Pub. No.: US 2005/0136417 A1
(43) Pub. Date: Jun. 23, 2005

(54) Amplification of nucleic acids

(75) Inventors: Kyle B. Cole, Stanford, CA (US); Vivi Truong, Mountain View, CA (US); Glenn H. McGall, Palo Alto, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/796,323

(22) Filed: Mar. 9, 2004

Related U.S. Application Data
(60) Provisional application No. 60/531,130, filed on Dec. 19, 2003.

(51) Int. Cl. C12Q 1/68; C12P 19/34

(52) U.S. Cl. 435/6; 435/91.2

(57) ABSTRACT

Methods and kits for amplifying nucleic acids are provided. Double stranded cDNA with uridine residues incorporated into one strand is synthesized and the uridine containing strand is nicked at uridine residues. The DNA is extended from the nicks in the presence of dUTP using a strand displacing enzyme. Repeated cycles of nicking and extension result in amplification of the nucleic acid. Methods are also provided for analysis of the above sample by hybridization to an array, which may be specifically designed to interrogate the collection of target sequences for particular characteristics, such as, for example, the presence or absence of one or more polymorphisms or the presence or absence of a transcript.

### RELATED APPLICATIONS

The present application claims priority to U.S. Provisional Application No. 60/531,130 filed Dec. 19, 2003, the entire disclosure of which is incorporated herein by reference in its entirety.

### FIELD OF THE INVENTION

The methods of the invention relate generally to amplification of nucleic acids by generating a nick and extending from the nick with a strand displacing polymerase.

### BACKGROUND OF THE INVENTION

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism.

### SUMMARY OF THE INVENTION

A method of amplifying nucleic acid is disclosed. A nucleic acid sample is obtained and first strand cDNA is synthesized using the nucleic acid sample as template. Second strand cDNA is synthesized in a reaction mixture comprising dUTP. The second strand cDNA is nicked and extended from the nicks using a DNA polymerase in a reaction mixture comprising dUTP. Downstream fragments of the second strand cDNA are displaced by the DNA polymerase during extension. In a preferred embodiment the nicking and extension steps are repeated.

In a preferred embodiment the second strand cDNA is nicked by generating abasic sites in the second strand cDNA by incubation with uracil DNA glycosylase and cleaving the second strand cDNA at the abasic sites. In preferred embodiments the abasic sites are cleaved by incubation with an apurinic endonuclease, for example, Endonuclease IV, by incubation at high temperature or by incubation under alkaline conditions.

In preferred embodiments the DNA polymerase is a strand displacing enzyme, for example the Klenow fragment or phi29. Nicking and extension may be performed under isothermal conditions, for example, at about 37° C. and may be performed simultaneously in the same reaction tube.

In one embodiment extension is performed in the presence of a mixture of dTTP and dUTP. The ratio of the dTTP to the dUTP regulates the length of the amplification products. The ratio may be, for example, greater than 5 to 1, or greater than 10 to 1 or greater than 100 to 1. The ratio of dTTP to dUTP may be selected based on the desired frequency of dUTP incorporation. For example, assuming that dTTP and dUTP are used at equivalent rates by the polymerase, if the ratio of dTTP to dUTP is 5 to 1 then roughly 5 T's will be incorporated for every U that is incorporated. Varying the ratio of dTTP to dUTP may be used to control the average length of the single stranded fragments that are generated. Another factor that may be considered is the frequency of T in the sequence to be amplified as this will impact the frequency of U that may be incorporated and the length of the resulting amplified cDNA. Labeled nucleotides may also be incorporated into the amplified fragments, for example, biotin-dUTP, biotin-dCTP or biotin-dATP. Because of the position of the biotin attachment, the bio-dUTP is not recognized by UDG. Detectable labels may be, for example, fluorescent or chemiluminescent.

The nucleic acid sample may comprise DNA or RNA or mixtures thereof. The DNA may be genomic DNA or DNA that has been amplified by PCR. In one embodiment, genomic DNA is fragmented, ligated to adaptors with common priming sites and fragments of a selected size range are amplified by PCR to generate the nucleic acid sample to be amplified by the methods disclosed. The first strand cDNA synthesis may be primed by addition of an exogenous primer comprising oligo dT, a plurality of locus specific primers or random primers. First strand cDNA may be synthesized by an RNA dependent DNA polymerase or by a DNA dependent DNA polymerase.

The amplified fragments may be used, for example, for genotyping or gene expression profile monitoring. Labeled amplified fragments may be hybridized to an array of probes and hybridization patterns may be detected. The array of probes may comprise, for example, allele specific probes to a plurality of polymorphisms, probes to transcripts of selected genes, probes that are tiled throughout a genome or a portion of a genome, for example, a chromosome, or probes that tiled to interrogate all possible single nucleotide variations in a genomic region, for example, a resequencing array.

### BRIEF DESCRIPTION OF THE FIGURE

FIG. 1 shows a schematic of amplification using deoxyuracil repair amplification. DNA is nicked at the location of uracils. The nicked DNA has free 3′ hydroxyl groups that can be extended, resulting in displacement of the downstream cDNA. The steps of nicking at uracils and extending from the nick may be repeated to generate many copies of DNA.

### DETAILED DESCRIPTION OF THE PREFERRED EMBODIMENTS

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above. Samples may be isolated from any material suspected of containing nucleic acid sequences. The source of the material may be, for example, buccal swab, blood, bone marrow, saliva, sputum, feces, urine, skin, or hard tissues such as liver, spleen, kidney, lung, ovary, breast, skin etc. Samples may be derived from plants, soil or other materials suspected of containing biological organisms or nucleic acids.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes. See also, Fodor et al., Science 251 (4995), 767-73, 1991, Fodor et al., Nature 364 (6437), 555-6, 1993 and Pease et al. PNAS USA 91 (11), 5022-6, 1994 for methods of synthesizing and using microarrays.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com. Arrays that may be used include, for example, expression arrays, genotyping arrays, resequencing arrays, whole transcriptome arrays, whole genome arrays, exon arrays and splicing arrays.

The present invention also contemplates many uses for polymers attached to solid substrates. These uses include gene expression monitoring, profiling, library screening, genotyping and diagnostics. Gene expression monitoring, and profiling methods are shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 10/442,021, 10/013,598, and U.S. Pat. Nos. 5,856,092, 6,300,063, 5,858,659, 6,284,460, 6,361,947, 6,368,799 and 6,333,179. Additional methods of genotyping, complexity reduction and nucleic acid amplification are disclosed in U.S. Patent Application Nos. 60/508,418, 60/468,925, 60/493,085, Ser. Nos. 09/920,491, 10/442,021, 10/654,281, 10/316,811, 10/646,674, 10/272,155, 10/681,773 and 10/712,616 and U.S. Pat. No. 6,582,938. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89: 117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Ser. No. 10/389,194 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/063,559 (United States Publication No. U.S. 20020183936), 60/349,546, 60/376,003, 60/394,574 and 60/403,381.

b) Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12: 203 (1984), incorporated herein by reference.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about IM, more usually less than about 500 mM and preferably less than about 200 mM. Hybridization temperatures can be as low as 5.degree. C., but are typically greater than 22.degree. C., more typically greater than about 30.degree. C., and preferably in excess of about 37.degree. C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25 degrees-Celsius (° C.), e.g., 750 mM NaCl, 50 mM NaPhosphate, 5 mM EDTA, pH 7.4 (5×SSPE) and a temperature of from about 25 to about 30° C.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule to a particular nucleotide sequence or sequences under selected hybridization conditions, typically stringent conditions, when that sequence is present in a complex mixture (e.g., total cellular) DNA or RNA.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population, but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies. Arrays comprising all possible probes sequences of a given length are disclosed in U.S. Pat. No. 6,582,908.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of silica based supports, like glasses, fused quartz, beads, resins, gels, microspheres, or other geometric configurations. See U.S. Pat. No. 5,744,305 for exemplary substrates.

Polymerases useful in this method include those that will initiate 5′ to 3′ polymerization at a nick site. The polymerase preferably should displace the polymerized strand downstream from the nick, and preferably lacks substantial 5′ to 3′ exonuclease activity. Enzymes that may be used include, for example, the Klenow fragment of DNA polymerase I, Bst polymerase large fragment, Phi29 and others. DNA Polymerase I Large (Klenow) Fragment consists of a single polypeptide chain (68 kDa) that lacks the 5′->3′ exonuclease activity of intact E. coli DNA polymerase I, but retains its 5′->3′ polymerase, 3′->5′ exonuclease and strand displacement activities. The Klenow fragment has been used for strand displacement amplification (SDA). See, e.g., U.S. Pat. Nos. 6,379,888; 6,054,279; 5,919,630; 5,856,145; 5,846,726; 5,800,989; 5,766,852; 5,744,311;5,736,365; 5,712,124; 5,702,926; 5,648,211;5,641,633; 5,624,825; 5,593,867; 5,561,044; 5,550,025; 5,547,861; 5,536,649; 5,470,723; 5,455,166; 5,422,252; 5,270,184, all incorporated herein by reference. SDA is an isothermal in vitro method for amplification of nucleic acid. SDA initiates synthesis of a copy of a nucleic acid at a free 3′ OH that may be provided, for example, by a primer that is hybridized to the template. The DNA polymerase extends from the free 3′ OH and in so doing displaces the strand that is hybridized to the template leaving a newly synthesized strand in its place. Repeated nicking and extension with continuous displacement of new DNA strands results in exponential amplification of the original template.

Phi29 is another DNA polymerase with high strand displacing activity. Phi29 is a highly processive enzyme that is capable of extending long regions of DNA, for example, 10 kb fragments. Variants of phi29 enzymes may be used, for example, an exonuclease minus variant may be used. For additional information on phi29 see, for example, U.S. Pat. Nos. 5,100,050, 5,198,543 and 5,576,204.

Bst DNA polymerase is another polymerase that is known to have strand displacing activity. The enzyme is available from, for example, New England Biolabs. Bst is active at high temperatures and the reaction may be incubated, for example at about 65° C. The enzyme tolerates reaction conditions of 70° C. and below and can be heat inactivated by incubation at 80° C. for 10 minutes. For additional information see Mead, D. A. et al. (1991) BioTechniques, p.p. 76-87, McClary, J. et al. (1991) J. DNA Sequencing and Mapping, p.p. 173-180 and Hugh, G. and Griffin, M. (1994) PCR Technology, p.p. 228-229.

Any polymerase with strand displacing activity may be used. Examples of other enzymes that may be used include: exo minus Vent (NEB), exo minus Deep Vent (NEB), Bst (BioRad), exo minus Pfu (Stratagene), Pfx (Invitrogen), 9°Nₘ™ (NEB), Bca (Panvera), and other thermostable polymerases. Other characteristics of strand displacing enzymes that may be taken into consideration are described, for example, in U.S. Pat. No. 6,692,918.

Uracil-DNA Glycosylase (UNG or UDG) catalyzes the removal of uracil from single- and double-stranded DNA that has been synthesized in the presence of dUTP. The apyriminic sites formed by UNG are susceptible to cleavage by heat under alkaline conditions or by apurinic endonucleases, such as endonuclease IV. The enzyme, which may be purified from E. coli and is commercially available, excises uracil from dU-containing DNA by cleaving the N-glycosidic bond between the uracil base and the sugar phosphate backbone. The resulting abasic sites may be hydrolyzed by alkali-treatment, high temperature, or endonucleases that cleave specifically at abasic sites. UNG will not digest 3′-terminal dU, dUTP, primers labeled with biotin-dUTP, or digoxigenin-dUTP. Ribouracil residues in RNA are also unaffected by UNG. For additional information about UNG and methods of use see, for example, Duncan, B. K. (1981) in Boyer (ed.) The Enzymes, Academic Press pp 565-586, Lindahl et al. (1978) J. Biol. Chem. 252: 3286-3294, Stuart and Chambers (1987) Nucl. Acids Res. 15: 7451-7462, and Krokan et al. Oncogene. 2002 Dec. 16; 21(58): 8935-48.

E. coli Endonuclease V recognizes uracil in duplex DNA and cleaves the second (about 95% of the time) and third (about 5% of the time) phosphodiester bonds 3′ to the uracil in the strand with the mismatch closest to the 5′ end. Endonuclease V also cleaves DNA duplexes containing inosine, AP sites, urea residues, hairpin or unpaired loops, flap, mismatches and pseudo-Y structures.

E. coli Endonuclease IV specifically catalyzes the formation of single strand breaks at apurinic and apyriminic sites in DNA. It also removes 3′-blocking groups (e.g. 3′-phosphoglycolate and 3′-phosphate) from damaged ends of DNA. Endonuclease IV is a class II AP (apurinic/apyrimidic) endonuclease with an associated 3′-diesterase activity and no associated N-glycosylase activity. Endonuclease IV can remove phosphoglycoaldehyde, deoxyribose-5-phosphate, 4-hydroxy-2-pentanal, and phosphate groups from the 3′ ends of DNA. Endonuclease IV does not contain 3′ exonuclease activity. The enzyme has no magnesium requirement and is fully active in EDTA. The enzyme is further described in the following references: Ljungquist, S., et al., J. Biol. Chem., 252, 2808-2814, 1977, Levin, J. D., J. Biol. Chem., 263, 8066-8071, 1988, Demple, B. and Harrison, L.,: Annu. Rev. Biochem., 63, 915-948, 1994, and Levin, J. D. and Demple, B., Nucleic Acids Res., 24, 885-889, 1996.

C. Nucleic Acid Amplification using Uracil DNA Glycosylase and Nicking

Nucleic acid amplification has extensive applications in gene expression profiling, genetic testing, diagnostics, environmental monitoring, resequencing, forensics, drug discovery and other areas. Nucleic acid samples may be derived, for example, from total nucleic acid from a cell or sample, total RNA, genomic DNA or mRNA. Preparations of total RNA or mRNA typically represent the transcribed regions of the genome. Many methods of analysis of nucleic acid may employ methods of amplification of the nucleic acid sample prior to analysis. Amplification methods may also be used to enrich an amplified sample for sequences of interest by preferential amplification of selected sequences. This may be accomplished, for example, by including a locus specific amplification step.

In one embodiment of the invention, methods are provided for isothermal amplification of target nucleic acid. The methods employ a cDNA synthesis step where uridine is incorporated into one strand of the cDNA, a nicking step in which one strand of a double stranded DNA is cleaved while the other strand is left in tact, and an extension step where DNA strands are displaced. The methods preferably employ multiple rounds of nicking followed by extension of the 3′ hydroxyl generated by the nicking with strand displacement. In a preferred embodiment nicking may be accomplished by treating the DNA with uracil DNA glycosylase (UDG) to generate abasic sites where uridine has been incorporated and then cleaving at the abasic sites by, for example, treatment with an AP endonuclease, alkaline treatment, heat treatment or a combination of treatments. In many embodiments a DNA polymerase having strand displacing activity which is preferably lacking 5′-3′ exonuclease activity (such as the DNA Polymerase I Large (Klenow) Fragment or similar enzymes) is used. See also U.S. patent application Ser. No. 10/318,692 which is incorporated herein by reference in its entirety.

In one embodiment target sequences are amplified from a complex mixture of sequences. The target sequences may contain a sequence of interest for further analysis, for example, a polymorphism, a gene of interest, or a splice variant of interest. Target sequences may be selected because they contain a sequence of interest such as a polymorphism or a gene of interest, for example, target sequences may be selected because they are from a gene whose expression is known to be altered in samples from individuals with a given disease or condition, genes that are known to be alternatively spliced, or genes that have polymorphisms of interest. Target sequences may be preferentially amplified by, for example, using locus specific primers to synthesize cDNA or by methods such as those described in U.S. patent application Ser. No. 10/681,773. For many nucleic acid analysis methods it is useful to amplify the sample to improve detection. For some methods it may be useful to amplify the sample by methods that result in enrichment of selected target sequences. The target sequences may be sequences that will be analyzed by downstream detection methods. For example, a genomic sample may be amplified by a method that enriches for a subset of selected target sequences and those selected target sequences may be detected by hybridization to an array of probes that are designed to detect the selected target sequences or to detect features, for example, polymorphisms, in the selected target sequences.

Nucleic acids amplified by the disclosed methods may be labeled and analyzed by hybridization to an array of probes, for example, the Affymetrix HU133 Plus 2.0 array which contains probes for analysis of over 47,000 transcripts. Comparable arrays are available to analyze expression from a variety of different organisms and arrays can be custom designed for an organism of interest or for a collection of transcripts of interest.

Nucleic acids amplified by the disclosed methods may also be analyzed on genotyping arrays which use allele specific hybridization methods to determine the genotype of SNPs or other polymorphisms. One example of such an array is the Affymetrix Mapping 10K Array. The array is designed to interrogate the genotype of over 10,000 human SNPs in a single assay. The genomic DNA is amplified using a whole genome sampling approach in which DNA is fragmented with a selected enzyme, ligated to a common adapter, amplified by PCR using a single primer, fragmented, labeled and hybridized to a genotyping array which contains probes to interrogate pre-selected SNPs. The current methods could also be used to amplify genomic DNA or in combination with the whole genome sampling approach described above.

In some embodiments mRNA is amplified by the disclosed methods and the resulting cDNA is analyzed to determine which mRNAs were present in the starting sample and at what level transcripts were present. Expression profiles may be generated from samples. The levels may be determined as relative to other transcripts, for example relative to a set of standard transcripts that should be present at relatively constant levels, for example, housekeeping genes. In another embodiment the amplification product is analyzed to determine the genotype of mRNAs in a sample. Some biological phenomena, for example, imprinting result in one copy of a gene being expressed in a diploid organism or tissue, while the second copy is not expressed. If the two alleles are distinguishable, for example if they have one heterozygous SNP, then it is possible to determine which of the two genes is being expressed by analyzing the mRNA. Failure of imprinting, where both alleles are expressed instead of just one, may also be detected.

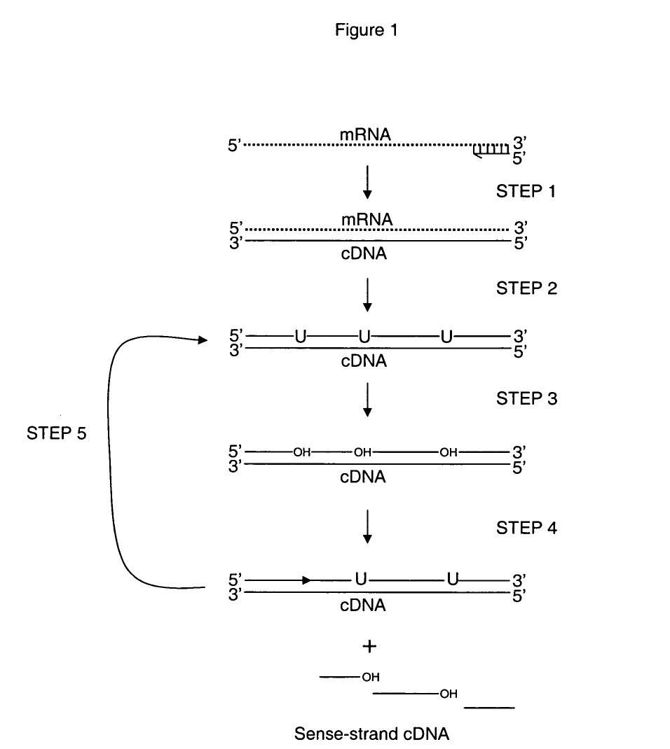

In one embodiment, shown in FIG. 1, mRNA sequences are amplified to produce many DNA copies of the mRNA. The amplified DNA is sense in orientation to the mRNA. In step 1 first strand cDNA is synthesized, for example using a primer comprising oligo dT or random primers. In step 2 second strand cDNA is synthesized in the presence of dUTP so that uridine is incorporated into the second strand cDNA. In step 3 the double stranded cDNA is incubated under conditions that result in nicking at positions where uridine is incorporated. Step 3 may comprise incubation with UDG which generates abasic sites in the second strand cDNA where uridines were incorporated followed by cleavage of the second strand cDNA at the abasic sites by, for example, treatment with an apurinic endonuclease, such as endonuclease IV, or by heat treatment. In another embodiment step 3 comprises incubation with an endonuclease that cleaves at uridines, for example, Endonuclease V. Step 3 generates free 3′ hydroxyl groups in the second strand cDNA where uridines were incorporated and the hydroxyls can be extended using the first strand cDNA as template (step 4). Downstream segments of the second strand cDNA may be displaced. Extension of the nicks may, for example, with Klenow exonuclease minus, or Phi 29. Extension of the free 3′ hydroxyl groups may be done in the presence of dUTP and the cycle of cleavage and extension with displacement may be repeated (step 5) a plurality of times to generate amplified sense-strand cDNA fragments.

In some embodiments the mRNA used in step 1 is present in a nucleic acid sample that has been isolated from a biological source such as a tissue sample. In other embodiments the mRNA that is used in step 1 may be an amplification product itself. For example, a nucleic acid sample could be amplified by another method that results in production of RNA and that RNA may be further amplified by the disclosed methods. Other methods of amplification that result in generation of amplified RNA include, for example, synthesizing RNA from a DNA template that contains an RNA polymerase promoter such as T7 RNA Polymerase.

In some embodiments the amplified sense DNA that is generated by the method, for example, in step 4 of FIG. 1, may be further amplified. Any amplification method may be used, for example, PCR with locus specific primers or amplification with random or degenerate primers.

In one embodiment, one or more steps of the method are isothermal. One or more of the enzymes used may be thermostable or thermolabile. In a preferred embodiment, extension is done in the presence of a detectable nucleotide, for example, biotin-dUTP, biotin-dCTP or biotin-dATP. The released sense strand cDNA may have incorporated detectable nucleotide and can subsequently be detected. In another embodiment the released sense strand cDNA may be end labeled.

In one embodiment, the ratio of dUTP to dTTP in the extension reaction is modulated to determine the average length of the amplified fragments. The length of fragments may be estimated by the distance between positions where uridines were incorporated and the ratio between dUTP and dTTP will determine how frequently a uridine is inserted opposite an A in the opposite strand. If the ratio of dTTP to dUTP is, for example, 10:1 then on average there will be 10 T's inserted for every U, the higher the ratio of dTTP to dUTP the less frequent the insertion of U and the longer the length of the amplified fragments.

One of skill in the art would appreciate that the amplification products generated by the methods are suitable for use with many methods for analysis of nucleic acids. In one preferred embodiment the amplified fragments are labeled with a detectable label and hybridized to an array of target specific probes, such as those available from Affymetrix under the brand name GeneChip®. Arrays are available, for example, for analysis of gene expression, and for genotyping. For additional information see, GeneChip Expression Analysis Technical Manual, 2003 and GeneChip Mapping Assay Manual, 2003. Oligonucleotide probes may also be immobilized on beads or optical fibers. In addition, the amplified fragments may be used for re-sequencing applications. Methods for resequencing using high density oligonucleotide probe arrays are disclosed in, e.g., U.S. patent application Ser. No. 10/028,482, which is incorporated by reference.

In another embodiment RNA may be amplified using an enzyme that nicks DNA that is part of an RNA:DNA duplex. The steps of the method may be as follows: isolate an RNA containing sample, make first strand cDNA by hybridizing a primer (oligo dT, random primers or locus specific primers) to the RNA and extending the primer with a reverse transcriptase to generate RNA:DNA duplexes; incubate the duplexes with an enzyme such as duplex specific nuclease (DSN) or a related enzyme, the amount of DSN activity added should be sufficient for nicking at the desired frequency without degrading the DNA; and extend the cDNA from the nicks using the RNA as template and displacing the downstream cDNA fragments as the polymerase extends. A DNA polymerase with strand displacing activity and reverse transcriptase activity would be used to extend from the nicks. Klenow, for example, may be used because it has high strand displacement activity and significant RT activity. Any polymerase with sufficient RT activity and sufficient strand displacement activity could be used.

DSN is available, for example, from Evrogen. The enzyme is from the Kamchatka crab. It is specific for dsDNA or DNA in a DNA:RNA hybrid but does not cleave ssDNA. The enzyme has been used to identify SNPs as it can discriminate between perfectly matched short DNA-DNA duplexes (8-12 bp) and duplexes of the same length with a single mismatch. The activity of the DSN enzyme may be controlled, for example, by the amount of enzyme added, the buffer conditions or the temperature conditions. The Ph and temperature optimum for DSN are 7-8 and 55-65° C. with the enzyme being stable at temps below 75° C. In some embodiments the cleavage and extension reactions are performed simultaneously so that multiple rounds of amplification can take place. The polymerase may be chosen so that it functions under similar conditions as the DSN enzyme. For example a mesophilic polymerase such as Bst1 may be used. For additional information about the DSN enzyme see, for example, Shagin et al. Genome Res 12: 1935-1942 (2002).

### Example 1

### (deoxy-Uracil Repair Amplification)dURA

A model dsDNA template was created by annealing two 47 nt oligos, one of which contains a deoxy-uracil that will act as the endonuclease recognition site (see FIG. 2). Uracil-containing double-stranded cDNA template (ds-cDNA) was created by performing standard first-strand cDNA synthesis, followed by second-strand synthesis with dUTP in addition to dTTP, dGTP, dCTP and dATP. Two methods of amplification were used, one utilizing E. coli endonuclease IV and one utilizing endonuclease V. The endonuclease IV reaction contains the enzymes uracil DNA glycosylase (UDG) to create an apyriminic site, endonuclease IV to remove the phosphate backbone creating an extensible 3′-hydroyl and a strand-displacing DNA polymerase such as Klenow exo-. As DNA polymerase extends the nick and displaces the annealed strand, a dUTP is inserted regenerating the original nicking site. Multiple rounds of nicking and synthesis generate many single stranded copies of the DNA template. The endonuclease V reaction does not require UDG because endonuclease V recognizes uracil in dsDNA and nicks 2 to 3 nt to the 3′ side of the uracil site creating extendible 3′ hydroxyls.

The endonuclease IV reaction produced the expected product size of 20 nucleotides. If dUTP was removed from the reaction less of the 20 nt product was produced as expected because one copy of ssDNA can be created from each template molecule present in the reaction before the uracil sites are replaced with thymine and nicking ceases.

The products generated from the endonuclease V reaction were expected to be in the size range of 17 to 18 nt. Products in the range of 16 to 40 nt were observed suggesting that endonuclease V may nick either DNA strand leading to amplification of both template strands. This does not occur when dUTP is removed from the reaction and a product of approximately the expected size is generated.

### Example 2

To further test the reaction a uracil-containing ds-cDNA was created from a 2.2 kb control transcript (polyA Thr). Reverse transcription was primed with dT24-T7 primer and dUTP was incorporated during second strand synthesis. Products ranging in size from 10 to 2200 nt were expected. Using 50-100 ng of ds-cDNA template, at least 6-8 ug of purified amplified DNA product was obtained.

The majority of the observed product was approximately 2200 nt and single stranded. It was somewhat unexpected that most of the product would be full length with relatively little smaller product being generated. In many embodiments this result is advantageous, because, for example, it minimizes bias in amplification that may occur. Both UDG and endo IV scan processively along DNA identifying lesions. One possible explanation is that endo IV nicks at the first AP site, which would be near the 5′ end of the sense strand, and pauses there waiting for the DNA repair machinery. It has been reported that human endo IV pauses after the first or second AP site (Cary and Strauss, 1999. Biochemistry 38: 16553-16560).

For the dURA Protocol the following reagents were used: 10× Eco Pol buffer (New England Biolabs (NEB) (Provided with Klenow Exo-), Klenow Fragment (3′->5′ exo-) [50U/ul] (NEB, high concentration version), Endonuclease IV [2U/ul] (Epicentre, stock was diluted by ten fold for protocol) and Uracil-DNA Glycosylase, UDG, (NEB).

Assemble the following reaction components on ice: 100 ng template DNA in a total volume 18.5 ul, 3.0 ul 2 mM dNTPs, 3.0 ul 10×EcoPol buffer, 1.0 ul Endonuclease IV [0.2U/ul], 1.0 ul Klenow Exo-[50U/ul], 3.0 ul 0.2 mM dUTP and 0.5 ul UDG [2U/ul]. The total volume in the reaction is 30.0 ul. Incubate at 37° C. for 4 hrs, heat inactivate enzymes at 75° C. for 20 min. and hold at 4° C. or store on ice.

5 ul of each reaction was analyzed on a 1.5% agarose gel with ethidium bromide at 120V for 1 hr. Alternatively, 1 ul of each reaction may be separated on a denaturing TBE-Urea gel and stained with SYBR Gold.

### CONCLUSION

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

1. A method of amplification of a nucleic acid sample comprising:
(a) obtaining a nucleic acid sample;
(b) contacting the nucleic acid sample with the appropriate reagents to synthesize a first strand cDNA;
(c) synthesizing a second strand cDNA in a reaction mixture comprising dUTP;
(d) nicking the second strand cDNA at one or more positions where dUTP was incorporated to generate one or more nicks; and
(e) extending the second strand cDNA from the one or more nicks in a reaction mixture comprising dUTP and a DNA polymerase with strand displacing activity, wherein downstream fragments of the second strand cDNA are displaced.

2. The method of claim 1 wherein steps (d) and (e) are performed simultaneously in a single reaction.

3. The method of claim 1 wherein step (d) comprises: generating abasic sites in the second strand cDNA and cleaving the second strand cDNA at the abasic sites.

4. The method of claim 3 wherein the abasic sites are generated by incubating with a uracil DNA glycosylase enzyme.

5. The method of claim 3 wherein the step of cleaving the second strand cDNA at the abasic sites comprises incubating the second strand cDNA with an apurinic endonuclease.

6. The method of claim 5 wherein the apurinic endonuclease is Endonuclease IV

7. The method of claim 3 wherein the step of cleaving the second strand cDNA at abasic sites comprises incubating the second strand cDNA at high temperature.

8. The method of claim 3 wherein the step of cleaving the second strand cDNA at abasic sites comprises incubating the second strand cDNA under alkaline conditions.

9. The method of claim 1 wherein first strand cDNA is synthesized in the presence of an RNA dependent DNA polymerase and second strand cDNA is synthesized in the presence of a DNA dependent DNA polymerase.

10. The method of claim 1 wherein the strand displacing DNA polymerase is selected from the group consisting of the Klenow fragment, Bst and phi29.

11. The method of claim 1 wherein the DNA polymerase is a phi29 variant that has reduced exonuclease activity.

12. The method of claim 1 wherein steps (d) and (e) are performed under isothermal conditions.

13. The method of claim 1 wherein steps (d) and (e) are performed at 37° C.

14. The method of claim 1 wherein Endonuclease V is used to nick the second strand cDNA in step (d).

15. The method of claim 1 wherein the reaction mixture of step (c) further comprises dTTP and the ratio of dTTP to dUTP in the starting mixture is greater than about 5 to 1.

16. The method of claim 1 the reaction mixture of step (e) further comprises a labeled nucleotide.

17. The method of claim 16 wherein the labeled nucleotide is biotin-dATP.

18. The method of claim 1 wherein the first strand cDNA is synthesized by a method comprising: hybridizing at least one primer to the nucleic acid sample and extending the primer with a polymerase.

19. The method of claim 18 wherein the nucleic acid sample comprises RNA and the polymerase is an RNA dependent DNA polymerase.

20. The method of claim 18 wherein the at least one primer comprises a 3′ oligo dT portion.

21. The method of claim 18 wherein the at least one primer comprises a mixture of primers of random sequence wherein the primers are of a common length and the length is between 6 and 15 nucleotides.

22. A method of detecting a target sequence in a nucleic acid sample comprising a complex mixture of sequences comprising:
(a) amplifying the nucleic acid sample by the method of claim 1;
(b) labeling the nucleic acids in the amplified nucleic acid sample with a detectable label;
(c) hybridizing the labeled, amplified nucleic acids to an array of probes comprising at least one probe that is perfectly complementary to the target sequence over the length of the probe;
(d) detecting a hybridization pattern; and,
(e) determining if the target sequence is present or absent based on the hybridization pattern.

23. The method of claim 21 wherein the label is biotin.

24. The method of claim 1 wherein the nucleic acid sample comprises RNA and first strand cDNA is synthesized using an RNA dependent DNA polymerase.

25. The method of claim 20 wherein first strand cDNA is synthesized by a primer comprising oligo dT.

26. The method of claim 20 wherein first strand cDNA synthesis is primed by a plurality of locus specific primers.

27. The method of claim 1 wherein the nucleic acid comprises genomic DNA.

28. The method of claim 23 wherein first strand cDNA synthesis is primed by a plurality of locus specific primers.

29. The method of claim 1 wherein the nucleic acid sample comprises adaptor ligated DNA fragments.

30. The method of claim 1 wherein the nucleic acid sample comprises adaptor ligated DNA fragments that have been amplified by PCR.

31. A method of genotyping at least one polymorphism from a sample comprising:
(a) obtaining a nucleic acid sample;
(b) contacting the nucleic acid sample with the appropriate reagents to synthesize a first strand cDNA from the nucleic acid sample;
(c) synthesizing a second strand cDNA in a reaction mixture comprising dUTP;
(d) nicking the second strand cDNA at one or more positions where dUTP was incorporated to generate one or more nicks in the second strand cDNA; and
(e) extending the second strand cDNA from the one or more nicks in a reaction mixture comprising dUTP and a DNA polymerase with strand displacing activity, wherein downstream fragments of the second strand cDNA are displaced by the DNA polymerase to generate displaced fragments;
(f) labeling the displaced fragments with a detectable label;
(g) hybridizing the labeled displaced fragments to an array of probes comprising at least one probe that is perfectly complementary to the target sequence over the length of the probe;
(a) detecting a hybridization pattern; and,
(b) determining if the target sequence is present or absent based on the hybridization pattern.

32. The method of claim 31 wherein the array of probes comprises a plurality of genotyping probe sets wherein each probe set comprises a first probe that is perfectly complementary to a first allele of a SNP and a second probe that is perfectly complementary to a second allele of the SNP wherein the first and second probes are between 20 and 50 nucleotides in length and the central position of each probes is complementary to the polymorphic position of the SNP.

33. A kit for amplifying nucleic acids comprising: a strand displacing DNA polymerase, dUTP, and uracil DNA glycosylase.

34. The kit of claim 33 further comprising Endonuclease IV.

35. The kit of claim 33 further comprising an RNA dependent DNA polymerase and a primer.

36. A kit for amplifying nucleic acids comprising: a strand displacing DNA polymerase, dUTP, and Endonuclease V.

37. The kit of claim 36 further comprising an RNA dependent DNA polymerase and a primer.
